(19) United States
(12) Patent Application Publication
(10) Pub. No.: US 2026/0007591 A1
(43) Pub. Date: Jan. 8, 2026

(54) NIMODIPINE COMPOSITION FOR INJECTION

(71) Applicant: BICAZER BIOTECHNOLOGY (GUANGZHOU) CO., LTD, Guangzhou (CN)

(72) Inventors: Gang WANG, Guangzhou (CN); Xiangyu DONG, Guangzhou (CN); Li LI, Guangzhou (CN); Yingting XU, Guangzhou (CN)

(73) Assignee: BICAZER BIOTECHNOLOGY (GUANGZHOU) CO., LTD, Guangzhou (CN)

(21) Appl. No.: 18/897,066

(22) Filed: Sep. 26, 2024

(30) Foreign Application Priority Data
Jul. 5, 2024 (CN) 202410898554.1

(51) Int. Cl. A61K 9/00; A61K 31/4418; A61K 47/10; A61K 47/32; A61K 47/40

(52) U.S. Cl. CPC A61K 9/0019; A61K 31/4418; A61K 47/10; A61K 47/32; A61K 47/40

(57) ABSTRACT

A nimodipine composition, comprising nimodipine or a pharmaceutically acceptable salt of nimodipine, cyclodextrin, stabilizer 1, stabilizer 2, and no more than 1% (w/w) organic solvent in the combination. A preparation method for nimodipine for injection, wherein the nimodipine for injection is prepared from the composition and a solvent, and the solvent is selected from ethanol and/or water. The mass ratio of sulfobutyl ether-β-cyclodextrin to nimodipine in the nimodipine composition for injection of the present disclosure is smaller, which can be expected to reduce the safety hazards caused by sulfobutyl ether-β-cyclodextrin. The ethanol proportion of the nimodipine composition for injection of the present disclosure is significantly reduced compared to Nimotop, avoiding the toxicity problems caused by the use of large amounts of organic solvents and improving patient compliance.

### CROSS-REFERENCE TO RELATED APPLICATIONS

This application claims priority to Chinese Patent Application Ser. No. CN202410898554.1 filed on 5 Jul. 2024.

### TECHNICAL FIELD

The present disclosure provides a dilution-stable nimodipine composition with extremely low organic solvent proportions, suitable for continuous intravenous administration.

### BACKGROUND

Nimodipine, the chemical name being isopropyl-2-methoxyethyl-1,4-dihydro-2,6-dimethyl-4 (3-nitrophenyl)-3,5-pyridine dicarboxylate, has vasodilatory properties, and is clinically used for the prevention and treatment of ischemic nerve damage caused by cerebral vasospasm after subarachnoid hemorrhage, as well as age-related brain dysfunction, migraine, sudden deafness, etc. The dosage forms of nimodipine used in clinical practice include tablets, capsules, injections, and injection powder.

Nimodipine has been approved in the United States as a Bayer oral liquid-filled capsule dosage form, but has not been approved for intravenous administration. However, in order to quickly and effectively control disease progression after subarachnoid hemorrhage (SAH), American physicians choose to extract the medication from the liquid-filled capsule and administer it intravenously to patients.

The FDA investigation shows that four patients who received this therapy died and five were seriously injured. The cause of death and injury may be due to the pharmacist's operation of extracting the medication from the capsule with high concentration, resulting in inaccurate volume and causing dosage errors.

Due to its rapid onset, nimodipine injection has unparalleled advantages over other dosage forms in the clinical treatment of cerebrovascular diseases. Europe has approved Bayer's intravenous injection formulation Nimotop®. Due to the insolubility of nimodipine in water, ethanol (23.7%) and PEG400 (17%) are used as solubilizers for nimodipine injection to increase its solubility. Although ethanol is an acceptable non-aqueous solvent for injection administration, the dosage should not be too large. When directly infused, the ethanol proportion is 23.7% and the non-aqueous solvent proportion is 40.7%, which can cause pain and irritation at the injection site. In addition, long-term intravenous infusion in clinical practice (often requiring more than 24 hours of infusion time) can cause significant vascular irritation and easily lead to phlebitis.

Nemotop is very prone to crystal precipitation when diluted directly. To reduce the concentration of the non-aqueous solvent in the intravenous infusion, Nemotop can be administered intravenously in a ratio of approximately 1:4 using a three-way valve with 5% glucose, 0.9% sodium chloride, sodium lactate Ringer's solution, magnesium-containing sodium lactate Ringer's solution, dextran 40 solution, or 6% HAES polyoxy-2-hydroxyethyl starch simultaneously. However, this infusion method poses significant safety risks. The infusion speed and mixing ratio are difficult to control, which can easily lead to imperceptible drug crystallization. The operation process is cumbersome and the costs of medication are high. Therefore, the compliance of medication in clinical practice is poor and the risk is high.

The large amount of ethanol in Nemotop is harmful to people with alcohol poisoning and impaired alcohol metabolism, as well as pregnant and lactating women.

For patients who find it difficult or impossible to swallow, intravenous administration route formulation of nimodipine is an essential clinical need.

Chinese patent CN 116889552 A discloses a lyophilized composition of nimodipine. In order to reconstruct or dilute the solution and stabilize it for 60 hours to meet clinical needs, the mass ratio of cyclodextrin to nimodipine must be as high as 500:1; the excipient ratio below 500 cannot meet the requirement of 60 hours of clinical use; if the excipient ratio is below 400, the dilution stability time is 36-48 hours; and the dilution stability time for the excipient ratio below 300 is less than 12 hours. CN 116889552 A discloses in [0367] that in order to address the dilution stability of nimodipine injection and meet the needs of clinical use for more than 60 hours, the mass ratio of sulfobutyl ether-β-cyclodextrin to nimodipine has been significantly improved to ≥500.

The present disclosure has found through research that a mass ratio of 100-300 of sulfobutyl ether-β-cyclodextrin to nimodipine can also meet the needs of clinical use for more than 60 hours.

Therefore, in order to improve the poor compliance of the current clinical use of nimodipine injection, it is of urgent clinical significance to reduce the proportion of the non-aqueous solvent in Nemotop formulation, lower the excipient ratio, and improve the dilution stability.

### SUMMARY

The objective of the present disclosure is to provide a nimodipine composition in response to the above-mentioned shortcomings of the prior art.

Another objective of the present disclosure is to provide a preparation method for nimodipine for injection.

Yet another objective of the present disclosure is to provide a prepared nimodipine for injection.

Based on the above-mentioned and other objectives, the present disclosure provides a nimodipine formulation with low exposure to non-aqueous solvent and ethanol. Low exposure to non-aqueous solvent and ethanol refers to the presence of the non-aqueous solvent and ethanol accounting for less than 1% (w/w) by weight in the composition.

The present disclosure provides a reconstituted or diluted nimodipine formulation with low exposure to non-aqueous solvent and ethanol. Low exposure to non-aqueous solvent and ethanol refers to the presence of the non-aqueous solvent and ethanol accounting for less than 1% (w/v) by weight in the reconstruction or dilution solution. In some embodiments, the weight of the non-aqueous solvent and ethanol in the reconstruction or dilution solution is less than 0.1% (w/v). In some embodiments, the weight of the non-aqueous solvent and ethanol in the reconstruction or dilution solution is less than 0.01% (w/v).

The present disclosure provides a nimodipine formulation which is not easy to crystallize during clinical infusion. Not easy to crystallize refers to that the nimodipine formulation can remain stable at room temperature for 8 hours without crystallization after reconstruction or dilution with water for injection, physiological saline, glucose, or other infusion solutions to a concentration of 0.04-1 mg/mL of nimodipine. In some embodiments, the nimodipine formulation can remain stable at room temperature for 24 hours without crystallization after reconstruction or dilution to a concentration of 0.04-1 mg/mL of nimodipine. The nimodipine formulation can remain stable at room temperature for 72 hours without crystallization after reconstruction or dilution to a concentration of 0.04-1 mg/mL of nimodipine.

The nimodipine formulation of the present disclosure uses cyclodextrin to improve the solubility and the reconstitution/dilution stability.

The objective of the present disclosure can be achieved through the following technical solutions:
a nimodipine composition, comprising nimodipine or a pharmaceutically acceptable salt of nimodipine, cyclodextrin, stabilizer 1, stabilizer 2, and no more than 1% (w/w) organic solvent in the combination.

In some embodiments, the weight of the non-aqueous solvent and ethanol in the composition is less than 0.1% (w/w). In some embodiments, the weight of the non-aqueous solvent and ethanol in the composition is less than 0.01% (w/w).

As a preferred embodiment of the present disclosure, the cyclodextrin is sulfobutyl-β-cyclodextrin, and the mass ratio of sulfobutyl-β-cyclodextrin to nimodipine is 100-300:1. During the preparation process (such as rotary evaporation, bringing to a constant volume, etc.) after dissolution, it should be clear and transparent, without crystal precipitation or other solid forms.

As a further preferred embodiment of the present disclosure, the cyclodextrin is sulfobutyl-β-cyclodextrin, and the mass ratio of sulfobutyl-β-cyclodextrin to nimodipine is 100-200:1 or 200-300:1.

In some examples of the present disclosure, the mass ratio of sulfobutyl-β-cyclodextrin to nimodipine is 100:1, 150:1, 160:1, 170:1, 180:1, 190:1, 200:1, 210:1, 220:1, 230:1, 240:1, 250:1, 260:1, 270:1, 280:1, 290:1, and 300:1.

As a preferred embodiment of the present disclosure, the stabilizer 1 is ethanol, the stabilizer 2 is PVPk12, the content of ethanol is 0.01-0.1%, and the mass ratio of PVPk12 to nimodipine is 0-200:1.

As a preferred embodiment of the present disclosure, the amount of PVPk12 is 0.

As another preferred embodiment of the present disclosure, the mass ratio of PVPk12 to nimodipine is 1-200:1, preferably 1-100:1, further preferably 10-50:1, and even further preferably 15-25:1.

In some examples of the present disclosure, the mass ratio of PVPk12 to nimodipine is 15:1, 20:1, and 25:1.

As a preferred embodiment of the present disclosure, the mass ratio of sulfobutyl-β-cyclodextrin:PVPk12:nimodipine is 100-300:10-30:1, preferably 150-300:10-50:1, further preferably 150-300:15-25:1, and even further preferably 150-200:15-25:1, or 150-200:15-25:1.

In some examples of the present disclosure, the mass ratio of sulfobutyl-β-cyclodextrin:PVPk12:nimodipine is 100:20:1, 150:20:1, 200:20:1, and 300:20:1.

A preparation method for nimodipine for injection, wherein the nimodipine for injection is prepared from the composition and a solvent, and the solvent is selected from ethanol and/or water.

As a preferred embodiment of the present disclosure, the preparation method comprises the following steps:
1) weighing 1 part of nimodipine, and dissolving in 500-1000 parts of ethanol to obtain an ethanol solution of nimodipine;
2) weighing 100-300 parts of sulfobutyl-β-cyclodextrin and 0-200 parts of stabilizer 2, and dissolving in 1000 parts of water to obtain an aqueous solution of sulfobutyl-β-cyclodextrin;
3) mixing the ethanol solution of nimodipine and the aqueous solution of sulfobutyl-β-cyclodextrin, stirring at an appropriate temperature to obtain clear intermediate 1; and
4) subjecting the clear intermediate 1 to rotary evaporation to remove ethanol, adjusting the concentration to obtain clear intermediate 2, filtering, packing, freeze-drying, and encapsulating to obtain nimodipine for injection.

As a preferred embodiment of the present disclosure, the stirring temperature in step 3) is 30-80° C., preferably 40-80° C., and further preferably 50-60° C.

As a preferred embodiment of the present disclosure, the ethanol concentration in the clear intermediate 2 in step 4) is 0.1-1%.

A nimodipine formulation for injection prepared by the preparation method according to the present disclosure.

Advantages of the present disclosure:
1) The mass ratio of sulfobutyl ether-β-cyclodextrin to nimodipine in the nimodipine composition for injection of the present disclosure is smaller, which can be expected to reduce the safety hazards caused by sulfobutyl ether-β-cyclodextrin.
2) The ethanol proportion of the nimodipine composition for injection of the present disclosure is significantly reduced compared to Nimotop, avoiding the toxicity problems caused by the use of large amounts of organic solvents and improving patient compliance.
3) The dilution stability of the nimodipine composition for injection of the present disclosure is improved. Nimodipine for injection can be directly reconstituted with physiological saline, 5% glucose injection, and water for injection to 0.04-0.2 mg/mL without the need for a three-way valve, improving safety and convenience of use.

In summary, the present disclosure provides a nimodipine for injection containing sulfobutyl ether-β-cyclodextrin, which can significantly reduce the proportion of cyclodextrin and the proportion of organic phase, significantly improve the dilution stability of nimodipine, and enhance patient compliance and clinical convenience, and has excellent clinical application prospects.

### DETAILED DESCRIPTION

In the following examples, unless otherwise specified, all water is water for injection.

### Example 1 Selection of Cyclodextrin Ratio

### 1. Prescription

TABLE 1.1
Prescription design

| | | SBECD | Ethanol | Water |
|---|---|---|---|---|
| | API (mg) | (mg) | (mL) | (mL) |
| Prescription | 100 | 10000 | 100 | 100 |
| 1.1 | | | | |
| Prescription | 100 | 15000 | 100 | 100 |
| 1.2 | | | | |
| Prescription | 100 | 20000 | 100 | 100 |
| 1.3 | | | | |
| Prescription | 100 | 25000 | 100 | 100 |
| 1.4 | | | | |
| Prescription | 100 | 30000 | 100 | 100 |
| 1.5 | | | | |
| Prescription | 100 | 40000 | 100 | 100 |
| 1.6 | | | | |
| Prescription | 100 | 50000 | 100 | 100 |
| 1.7 | | | | |

### 2. Preparation Process

API was dissolved in ethanol, SBECD was dissolved in water, and the two solutions were mixed and stirred at 50° C. for 3 hours to obtain clear intermediate 1. Ethanol was removed by rotary evaporation at 40° C., and water was added to 1 mg/mL to obtain clear intermediate 2. The clear intermediate 2 was filtered and packed, and freeze-dried.

### 3. Freeze-Dried Powder Redissolution Stability

The freeze-dried powder was redissolved with physiological saline to API concentrations of 1, 0.2, and 0.04 mg/mL. The stability of the solution was observed. At time points 8, 12, 24, 48, and 72 hours, if crystal precipitation was observed, it was recorded as “+”, and if no crystal precipitation was observed, it was recorded as “−”.

TABLE 1.2
Freeze-dried powder redissolution stability

| | | 8 h | 12 h | 24 h | 48 h | 72 h |
|---|---|---|---|---|---|---|
| Prescription | 1 | − | − | + | + | + |
| 1.1 | mg/mL | | | | | |
| | 0.2 | − | − | + | + | + |
| | mg/mL | | | | | |
| | 0.04 | − | − | − | + | + |
| | mg/mL | | | | | |
| Prescription | 1 | − | − | − | − | − |
| 1.2 | mg/mL | | | | | |
| | 0.2 | − | − | − | − | − |
| | mg/mL | | | | | |
| | 0.04 | − | − | − | − | − |
| | mg/mL | | | | | |
| Prescription | 1 | − | − | − | − | − |
| 1.3 | mg/mL | | | | | |
| | 0.2 | − | − | − | − | − |
| | mg/mL | | | | | |
| | 0.04 | − | − | − | − | − |
| | mg/mL | | | | | |
| Prescription | 1 | − | − | − | − | − |
| 1.4 | mg/mL | | | | | |
| | 0.2 | − | − | − | − | − |
| | mg/mL | | | | | |
| | 0.04 | − | − | − | − | − |
| | mg/mL | | | | | |
| Prescription | 1 | − | − | − | − | − |
| 1.5 | mg/mL | | | | | |
| | 0.2 | − | − | − | − | − |
| | mg/mL | | | | | |
| | 0.04 | − | − | − | − | − |
| | mg/mL | | | | | |
| Prescription | 1 | − | − | − | − | − |
| 1.6 | mg/mL | | | | | |
| | 0.2 | − | − | − | − | − |
| | mg/mL | | | | | |
| | 0.04 | − | − | − | − | − |
| | mg/mL | | | | | |
| Prescription | 1 | − | − | − | − | − |
| 1.7 | mg/mL | | | | | |
| | 0.2 | − | − | − | − | − |
| | mg/mL | | | | | |
| | 0.04 | − | − | − | − | − |
| | mg/mL | | | | | |

The experimental results indicate that:
1) when the API concentration is 1 mg/mL and the ratio of API to SBECD is 1:100, the solution can be maintained for 12 hours without precipitation. Increasing the proportion of SBECD can maintain no precipitation for 72 hours.
2) The lower the API concentration after redissolution of the freeze-dried powder, the better the stability of the solution. Except for prescription 1.1, all other prescriptions can remain stable for 72 hours without crystallization at a concentration of 0.2 mg/mL, which can meet the clinical need for direct infusion of Nimotop. Except for prescription 1.1, all other prescriptions can remain stable for 72 hours without crystallization at a concentration of 0.04 mg/mL, which can meet the clinical need for infusion of Nimotop diluted via Y-tube.
3) Increasing the proportion of SBECD can improve the dilution stability of nimodipine for injection.

### Example 2 Selection of Ethanol Proportion of Intermediate 1

### 1. Prescription

TABLE 2.1
Prescription design

| | | SBECD | Ethanol | Water |
|---|---|---|---|---|
| | API (mg) | (mg) | (mL) | (mL) |
| Prescription | 100 | 30000 | 20 | 100 |
| 2.1 | | | | |
| Prescription | 100 | 30000 | 40 | 100 |
| 2.2 | | | | |
| Prescription | 100 | 30000 | 60 | 100 |
| 2.3 | | | | |
| Prescription | 100 | 30000 | 100 | 100 |
| 2.4 | | | | |

### 2. Preparation Process

API was dissolved in ethanol, SBECD was dissolved in water, and the two solutions were mixed and stirred at 50° C. for 3 hours to obtain clear intermediate 1. Ethanol was removed by rotary evaporation at 40° C., and water was added to 1 mg/mL to obtain clear intermediate 2. The clear intermediate 2 was filtered and packed.

### 3. Investigation of Stability of Intermediate 2

The stability of intermediate 2 solution was observed. At time points 8, 12, 24, 48, and 72 hours, if crystal precipitation was observed, it was recorded as “+”, and if no crystal precipitation was observed, it was recorded as “−”.

TABLE 2.2
Investigation of stability of intermediate 2

| | 8 h | 12 h | 24 h | 48 h | 72 h |
|---|---|---|---|---|---|
| Prescription | + | + | + | + | + |
| 2.1 | | | | | |
| Prescription | − | − | − | − | − |
| 2.2 | | | | | |
| Prescription | − | − | − | − | − |
| 2.3 | | | | | |
| Prescription | − | − | − | − | − |
| 2.4 | | | | | |

The experimental results indicate that when the ratio of ethanol to water in the prescription is 20:100, stable intermediate 2 solution cannot be obtained for further operation. Increasing the ethanol proportion can help improve the stability of the intermediate 2 solution.

### Example 3: Selection of Ethanol Concentration for Intermediate 2

### 1. Prescription

TABLE 3.1
Prescription design

| | | SBECD | Ethanol | Water |
|---|---|---|---|---|
| | API (mg) | (mg) | (mL) | (mL) |
| Prescription | 100 | 30000 | 100 | 100 |
| 3.1 | | | | |

### 2. Preparation Process

API was dissolved in ethanol, SBECD was dissolved in water, and the two solutions were mixed and stirred at 50° C. for 3 hours to obtain clear intermediate 1. Ethanol was removed by rotary evaporation at 40° C., and water was added to 1 mg/mL to obtain clear intermediate 2. Ethanol residue was detected. The clear intermediate 2 was filtered and packed, and freeze-dried.

By controlling the duration of rotary evaporation, intermediate 2 with different ethanol residues was obtained, and there are three ethanol residue standards: >1%, 0.1-1%, and <0.1%.

### 3. Investigation of Freeze-Dried Powder Redissolution Stability

The freeze-dried powder was redissolved with physiological saline to API concentrations of 1, 0.2, and 0.04 mg/mL. The stability of the solution was observed. At time points 8, 12, 24, 48, and 72 hours, if crystal precipitation was observed, it was recorded as “+”, and if no crystal precipitation was observed, it was recorded as “−”.

TABLE 3.2
Freeze-dried powder redissolution stability

| Ethanol | | | | | | |
|---|---|---|---|---|---|---|
| residue | | 8 h | 12 h | 24 h | 48 h | 72 h |
| >1% | 1 | − | + | + | + | + |
| | mg/mL | | | | | |
| | 0.2 | − | − | + | + | + |
| | mg/mL | | | | | |
| | 0.04 | − | − | − | + | + |
| | mg/mL | | | | | |
| 0.1-1% | 1 | − | − | − | − | − |
| | mg/mL | | | | | |
| | 0.2 | − | − | − | − | − |
| | mg/mL | | | | | |
| | 0.04 | − | − | − | − | − |
| | mg/mL | | | | | |
| <0.1% | 1 | − | − | + | + | + |
| | mg/mL | | | | | |
| | 0.2 | − | − | − | − | − |
| | mg/mL | | | | | |
| | 0.04 | − | − | − | − | − |
| | mg/mL | | | | | |

The experimental results indicate that residual ethanol in intermediate 2 can affect the stability of the solution when redissolved to an API concentration of 1 mg/mL. Ethanol residues above 1% or below 0.1% are not conducive to the stability of the solution after redissolution of the freeze-dried powder.

### Example 4: Selection of Multi-Component Mixture

### 1. Prescription

TABLE 4.1
Prescription design

| | API | SBECD | Ethanol | Water | PVPk12 |
|---|---|---|---|---|---|
| | (mg) | (mg) | (mL) | (mL) | (mg) |
| Prescription | 100 | 10000 | 100 | 100 | 0 |
| 4.1 | | | | | |
| Prescription | 100 | 10000 | 100 | 100 | 500 |
| 4.2 | | | | | |
| Prescription | 100 | 10000 | 100 | 100 | 1000 |
| 4.3 | | | | | |
| Prescription | 100 | 10000 | 100 | 100 | 2000 |
| 4.4 | | | | | |
| Prescription | 100 | 15000 | 100 | 100 | 2000 |
| 4.5 | | | | | |

### 2. Preparation Process

API was dissolved in ethanol, SBECD and PVPk12 were dissolved in water, and the two solutions were mixed and stirred at 50° C. for 3 hours to obtain clear intermediate 1. Ethanol was removed by rotary evaporation at 40° C., and water was added to 1 mg/mL to obtain clear intermediate 2. The clear intermediate 2 was filtered and packed, and freeze-dried.

### 3. Investigation of Stability of Intermediate 2

TABLE 4.2
Stability of intermediate 2

| | 8 h | 12 h | 24 h | 48 h | 72 h |
|---|---|---|---|---|---|
| Prescription | − | + | + | + | + |
| 4.1 | | | | | |
| Prescription | − | − | − | + | + |
| 4.2 | | | | | |
| Prescription | − | − | − | + | + |
| 4.3 | | | | | |
| Prescription | − | − | − | − | + |
| 4.4 | | | | | |
| Prescription | − | − | − | − | − |
| 4.5 | | | | | |

### 4. Investigation of Freeze-Dried Powder Dilution Stability

The freeze-dried powder was redissolved with physiological saline to API concentrations of 1, 0.2, and 0.04 mg/mL. The stability of the solution was observed. At time points 8, 12, 24, 48, and 72 hours, if crystal precipitation was observed, it was recorded as “+”, and if no crystal precipitation was observed, it was recorded as “−”.

TABLE 4.3
Freeze-dried powder dilution stability

| | | 8 h | 12 h | 24 h | 48 h | 72 h |
|---|---|---|---|---|---|---|
| Prescription | 1 | − | − | + | + | + |
| 4.1 | mg/mL | | | | | |
| | 0.2 | − | − | + | + | + |
| | mg/mL | | | | | |
| | 0.04 | − | − | − | + | + |
| | mg/mL | | | | | |
| Prescription | 1 | − | − | + | + | + |
| 4.2 | mg/mL | | | | | |
| | 0.2 | − | − | | + | + |
| | mg/mL | | | | | |
| | 0.04 | − | − | − | − | + |
| | mg/mL | | | | | |
| Prescription | 1 | − | − | + | + | + |
| 4.3 | mg/mL | | | | | |
| | 0.2 | − | − | − | + | + |
| | mg/mL | | | | | |
| | 0.04 | − | − | − | − | + |
| | mg/mL | | | | | |
| Prescription | 1 | − | − | − | + | + |
| 4.4 | mg/mL | | | | | |
| | 0.2 | − | − | − | + | + |
| | mg/mL | | | | | |
| | 0.04 | − | − | − | − | + |
| | mg/mL | | | | | |
| Prescription | 1 | − | − | − | − | − |
| 4.5 | mg/mL | | | | | |
| | 0.2 | − | − | − | − | − |
| | mg/mL | | | | | |
| | 0.04 | − | − | − | − | − |
| | mg/mL | | | | | |

### 3. Result Analysis

1) The observation results of stability of intermediate 2 showed that the addition of PVPk12 significantly improved the stability of intermediate 2, and with the increase of PVPk12 proportion, the stability gradually improved.
2) As the proportion of PVPk12 increases, the freeze-dried powder redissolution stability also improves to some extent.

### Example 5 Selection of Stirring Temperature

### 1. Prescription

TABLE 5.1
Prescription design

| | | | 50% | | |
|---|---|---|---|---|---|
| | API | SBECD | ethanol | PVPk12 | Temperature |
| | (mg) | (mg) | (mL) | (mg) | (° C.) |
| Prescription | 100 | 15000 | 200 | 2000 | 30 |
| 5.1 | | | | | |
| Prescription | 100 | 15000 | 200 | 2000 | 50 |
| 5.2 | | | | | |
| Prescription | 100 | 15000 | 200 | 2000 | 80 |
| 5.3 | | | | | |
| Prescription | 100 | 30000 | 200 | 2000 | 30 |
| 5.4 | | | | | |
| Prescription | 100 | 30000 | 200 | 2000 | 50 |
| 5.5 | | | | | |
| Prescription | 100 | 30000 | 200 | 2000 | 80 |
| 5.6 | | | | | |

### 2. Preparation Process

API was dissolved in ethanol, SBECD and PVPk12 were dissolved in water, and the two solutions were mixed and stirred at the prescribed temperature for 3 hours to obtain clear intermediate 1. Ethanol was removed by rotary evaporation at 40° C., and water was added to 1 mg/mL to obtain clear intermediate 2. The clear intermediate 2 was filtered and packed.

### 3. Investigation of Stability of Intermediate 2

The stability of intermediate 2 was observed. At time points, if crystal precipitation was observed, it was recorded as “+”, and if no crystal precipitation was observed, it was recorded as “−”.

TABLE 5.2
Stability of intermediate 2

| | 8 h | 12 h | 24 h | 48 h | 72 h |
|---|---|---|---|---|---|
| Prescription | − | − | − | + | + |
| 5.1 | | | | | |
| Prescription | − | − | − | − | − |
| 5.2 | | | | | |
| Prescription | − | − | − | − | − |
| 5.3 | | | | | |
| Prescription | − | − | − | − | + |
| 5.4 | | | | | |
| Prescription | − | − | − | − | − |
| 5.5 | | | | | |
| Prescription | − | − | − | − | − |
| 5.6 | | | | | |

The experimental results indicate that the stability of intermediate 2 prepared at 30° C. was poor, and as the temperature increased to 50° C., the stability of intermediate 2 could reach up to 72 hours.

### 4. Investigation of Freeze-Dried Powder Dilution Stability

The freeze-dried powder was redissolved with physiological saline to API concentrations of 1, 0.2, and 0.04 mg/mL. The stability of the solution was observed. At time points 8, 12, 24, 48, and 72 hours, if crystal precipitation was observed, it was recorded as “+”, and if no crystal precipitation was observed, it was recorded as “−”.

TABLE 5.3
Freeze-dried powder stability

| | | 8 h | 12 h | 24 h | 48 h | 72 h |
|---|---|---|---|---|---|---|
| Prescription | 1 | − | − | + | + | + |
| 5.1 | mg/mL | | | | | |
| | 0.2 | − | − | − | − | + |
| | mg/mL | | | | | |
| | 0.04 | − | − | − | − | + |
| | mg/mL | | | | | |
| Prescription | 1 | − | − | − | − | − |
| 5.2 | mg/mL | | | | | |
| | 0.2 | − | − | − | − | − |
| | mg/mL | | | | | |
| | 0.04 | − | − | − | − | − |
| | mg/mL | | | | | |
| Prescription | 1 | − | − | − | − | + |
| 5.3 | mg/mL | | | | | |
| | 0.2 | − | − | − | − | + |
| | mg/mL | | | | | |
| | 0.04 | − | − | − | − | − |
| | mg/mL | | | | | |
| Prescription | 1 | − | − | − | − | − |
| 5.4 | mg/mL | | | | | |
| | 0.2 | − | − | − | − | − |
| | mg/mL | | | | | |
| | 0.04 | − | − | − | − | − |
| | mg/mL | | | | | |
| Prescription | 1 | − | − | − | − | − |
| 5.5 | mg/mL | | | | | |
| | 0.2 | − | − | − | − | − |
| | mg/mL | | | | | |
| | 0.04 | − | − | − | − | − |
| | mg/mL | | | | | |
| Prescription | 1 | − | − | − | − | − |
| 5.5 | mg/mL | | | | | |
| | 0.2 | − | − | − | − | − |
| | mg/mL | | | | | |
| | 0.04 | − | − | − | − | − |
| | mg/mL | | | | | |

The experimental results indicate that the dilution stability of nimodipine for injection prepared at 30° C. was poor, and it was necessary to increase the amount of sulfobutyl-β-cyclodextrin to improve the stability. As the temperature was increased to 50° C., the dilution stability of nimodipine for injection was optimal. Although the dilution stability of nimodipine for injection prepared at 80° C. was better than that at 30° C., excessively high temperatures were not conducive to optimizing the dilution stability of nimodipine for injection.

### Example 6 Investigation of Dilution Stability with Different Solvents

Nimodipine for injection prepared in prescriptions 5.2 and 5.5 was taken, diluted with physiological saline, 5% glucose injection, and water for injection to clinical concentrations of 0.2 and 0.04 mg/mL, respectively, and the dilution stability at 8, 12, 24, 48, and 72 hours was investigated. If crystal precipitation was observed, it was recorded as “+”, and if no crystal precipitation was observed, it was recorded as “−”.

### Method for Determining the Content of Nimodipine for Injection:

1. Chromatographic conditions: Octadecylsilane bonded silica as the filler (C18 column 100×4.6 mm, 4 μm); methanol-tetrahydrofuran (80:20) as mobile phase A, water-tetrahydrofuran (80:20) as mobile phase B, mobile phase A:mobile phase B=(25:75) as mobile phase; the column temperature: 40° C.; the flow rate: 2.0 mL/min; the injection volume: 10 μL; the detection wavelength: 235 nm; and the analysis time: 15 minutes.
2. Solvent:methanol:tetrahydrofuran:water=20:20:60.
3. Preparation of reference solution: 10 mg of nimodipine reference was accurately weighed, placed in a 50 ml volumetric flask, dissolved in 5 ml of tetrahydrofuran, diluted to the mark with solvent, and shaken well to obtain the reference solution. 4. Preparation of test solution: 1.3 g of nimodipine for injection was accurately weighed, placed in a 20 mL brown volumetric flask, dissolved in solvent and brought to volume to the mark, and shaken well to obtain the test solution.

TABLE 6.1
Freeze-dried powder solvent dilution stability

| | | | | | | | 72 h |
|---|---|---|---|---|---|---|---|
| | | | | | | | content |
| Prescription 5.2 | | 8 h | 12 h | 24 h | 48 h | 72 h | (%) |
| Physiological | 0.2 | — | — | — | — | — | 99.60 |
| saline | mg/mL | | | | | | |
| | 0.04 | — | — | — | — | — | 99.75 |
| | mg/mL | | | | | | |
| 5% glucose | 0.2 | — | — | — | — | — | 100.04 |
| | mg/mL | | | | | | |
| | 0.04 | — | — | — | — | — | 100.03 |
| | mg/mL | | | | | | |
| Water for | 0.2 | — | — | — | — | — | 99.66 |
| injection | mg/mL | | | | | | |
| | 0.04 | — | — | — | — | — | 99.59 |
| | mg/mL | | | | | | |

| | | | | | | | 72 h |
|---|---|---|---|---|---|---|---|
| Prescription | | 8 | 12 | 24 | 48 | 72 | content |
| 5.5 | | h | h | h | h | h | (%) |
| Physiological | 0.2 | — | — | — | — | — | 99.59 |
| saline | mg/mL | | | | | | |
| | 0.04 | — | — | — | — | — | 99.66 |
| | mg/mL | | | | | | |
| 5% glucose | 0.2 | — | — | — | — | — | 100.12 |
| | mg/mL | | | | | | |
| | 0.04 | — | — | — | — | — | 100.12 |
| | mg/mL | | | | | | |
| Water for | 0.2 | — | — | — | — | — | 99.81 |
| injection | mg/mL | | | | | | |
| | 0.04 | — | — | — | — | — | 99.95 |
| | mg/mL | | | | | | |

### Experimental Results

The results of redissolution prescriptions 5.2 and 5.5 with different dilution solvents indicate that the nimodipine for injection in the present disclosure can be directly diluted to 0.2 mg/mL and 0.04 mg/mL, and can remain stable for up to 72 hours under different solvent conditions without crystal precipitation. The content is above 99%, which is sufficient to meet the needs of clinical use.

### Example 7 Investigation of High Temperature Stability

### 1. Experimental Methods

Nimodipine for injection was prepared according to prescription 5.5, and the content, related substances, and ethanol residue of nimodipine for injection were investigated.

### Method for Determining Related Substances of Nimodipine for Injection:

1. Chromatographic conditions: Octadecylsilane chemically bonded silica as the filler (C18 column 100×4.6 mm, 4 μm); methanol-tetrahydrofuran (80:20) as mobile phase A, water-tetrahydrofuran (80:20) as mobile phase B, mobile phase A:mobile phase B=(15:85) as mobile phase; the column temperature: 40° C.; the flow rate: 2.0 mL/min; the injection volume: 10 μL; detection wavelength: 235 nm; and the analysis time: 30 minutes.
2. Solvent:methanol:tetrahydrofuran:water=20:20:60.
3. Preparation of reference solution: 10 mg of nimodipine reference was accurately weighed, placed in a 50 ml volumetric flask, dissolved in 5 ml of tetrahydrofuran, diluted to the mark with solvent, and shaken well to obtain the reference solution.
4. Preparation of test solution: 1.3 g of nimodipine for injection was accurately weighed, placed in a 20 mL brown volumetric flask, dissolved in solvent and brought to volume to the mark, and shaken well to obtain the test solution.
5. Preparation of system suitability solution: Appropriate amounts of nimodipine and impurity I reference were taken, dissolved and diluted in a solvent to prepare a mixed solution containing approximately 200 μg and 1 μg per 1 ml, respectively.
6. Reference solution (impurity A): Impurity A reference was taken, accurately weighed, dissolved in mobile phase and diluted quantitatively to make a solution of 20 μg/ml. 1 ml of the solution was accurately measured, placed in a 100 ml volumetric flask, and diluted with solvent to the mark.

### Method for Determining Ethanol Residue of Nimodipine for Injection:

Chromatographic conditions: the initial temperature: 40° C., maintaining for 2 minutes; heating up at a rate of 3° C. per minute to 65° C., then at a rate of 25° C. per minute to 200° C., and maintaining for 10 minutes; a total of 25.7 minutes. The inlet temperature was 200° C., and the detector temperature was 220° C.

Headspace injection conditions: the constant temperature furnace temperature: 85° C.; the gas flow path temperature: 90° C.; the transmission line temperature: 100° C.; the flask pressurization pressure: 160 kPa; the flask constant temperature time: 20 minutes, the pressurization time: 1 minute, and the pressurization equilibrium time: 0.1 minute; the import time: 0.5 minute, the import balancing time: 0.1 minute; and the injection time: 1 minute.

Reference solution: 0.5 mL of anhydrous ethanol solution was precisely drawn, placed in a 10 mL volumetric flask containing a small amount of water, mixed well to continue to be diluted with water to the mark, and shaken well to obtain the standard mother liquor. 1 mL of mother liquor was accurately transferred, placed in a 100 mL volumetric flask, diluted with solvent and brought to volume to the mark, shaken well, and the solution was ready. 2 mL of each solution was taken into a headspace injection flask and sealed.

Test solution: 0.6 g of nimodipine for injection was weighed, placed in a 10 mL volumetric flask, dissolved in solvent and diluted to the mark, and shaken well to obtain the test solution. 2 mL of the solution was taken into a headspace injection flask and sealed.

### 2. Experimental Results

TABLE 7.1
Long-term stability investigation results of nimodipine for injection

| | | | | Total | Con- | Ethanol |
|---|---|---|---|---|---|---|
| | Duration | Impurity | Impurity | impurities | tent | residue |
| | (months) | A (%) | C (%) | (%) | (%) | (%) |
| 25° C. | 1 | 0.00 | 0.03 | 0.03 | 99.77 | 0.16 |
| 30° C. | | 0.00 | 0.03 | 0.03 | 99.78 | 0.18 |
| 40° C. | | 0.00 | 0.03 | 0.03 | 99.76 | 0.20 |
| 25° C. | 3 | 0.00 | 0.03 | 0.03 | 99.81 | 0.22 |
| 30° C. | | 0.00 | 0.03 | 0.03 | 99.81 | 0.12 |
| 40° C. | | 0.00 | 0.03 | 0.03 | 99.79 | 0.16 |
| 25° C. | 6 | 0.00 | 0.03 | 0.03 | 99.82 | 0.18 |
| 30° C. | | 0.00 | 0.03 | 0.03 | 99.81 | 0.22 |
| 40° C. | | 0.00 | 0.03 | 0.03 | 99.78 | 0.18 |
| 25° C. | 9 | 0.00 | 0.03 | 0.03 | 99.76 | 0.20 |
| 30° C. | | 0.00 | 0.02 | 0.02 | 99.75 | 0.20 |
| 40° C. | | 0.00 | 0.03 | 0.03 | 99.82 | 0.22 |
| 25° C. | 12 | 0.00 | 0.03 | 0.05 | 99.70 | 0.26 |
| 30° C. | | 0.00 | 0.03 | 0.05 | 99.81 | 0.12 |
| 40° C. | | 0.02 | 0.03 | 0.07 | 99.77 | 0.20 |

### 3. Result Analysis

In the long-term stability experiment of nimodipine for injection at three temperatures, the related substances of nimodipine remained almost unchanged, and the nimodipine for injection of the present disclosure has good stability.

### Comparison Example 1

The experimental results of Example 39 in CN 116889552 are described herein:

| 1) Excipient | |
|---|---|
| ratio | 2) Dilution stability situation |
| 3) 300 | 4) The content significantly decreases after 12 hours, |
| | and the stable time is less than 12 hours |
| 5) 350 | 6) There is almost no change in the content after 24 |
| | hours, and the stability time is greater than 24 hours |
| 7) 400 | 8) There is almost no change in the content after 36 |
| | hours, and the stability time is greater than 36 hours |
| | and less than 48 hours |
| 9) 450 | 10) The content almost changes after 48 hours, and the |
| | stability time is greater than 48 hours |
| 11) 500 | 12) The content almost changes after 60 hours, and the |
| | stability time is greater than 60 hours |
| 13) 600 | 14) The content almost changes after 60 hours, and the |
| | stability time is greater than 60 hours |
| 15) 700 | 16) The content almost changes after 60 hours, and the |
| | stability time is greater than 60 hours |

The disclosure of CN116889552 states that: “ . . . when the excipient ratio is ≥500, the dilution stability time of the nimodipine cyclodextrin inclusion complex can reach more than 60 hours, meeting the clinical use needs. Based on the above-mentioned reasons, we have increased the range of the mass ratio of sulfobutyl ether-β-cyclodextrin to nimodipine in the claims.”

From Comparative example 1, it can be determined that the nimodipine injection invented in CN116889552 can only meet the clinical use needs when the excipient ratio is higher than 500. Calculated based on 500 required in CN116889552, the excipient ratio in the present disclosure is only 30-60%, greatly reducing the amount of excipients used.

What is claimed is:

1. A nimodipine composition, comprising nimodipine or a pharmaceutically acceptable salt of nimodipine, cyclodextrin, stabilizer 1, stabilizer 2, and no more than 1% organic solvent in the combination.

2. The nimodipine composition according to claim 1, wherein the cyclodextrin is sulfobutyl-β-cyclodextrin, and the mass ratio of sulfobutyl-β-cyclodextrin to nimodipine is 100-300:1, and preferably, the mass ratio of sulfobutyl-β-cyclodextrin to nimodipine is 100-200:1 or 200-300:1.

3. The nimodipine composition according to claim 2, wherein the stabilizer 1 is ethanol, the stabilizer 2 is PVPk12, the content of ethanol is 0.01-0.1%, and the mass ratio of PVPk12 to nimodipine is 0-200:1.

4. The nimodipine composition according to claim 3, wherein the amount of PVPk12 is 0, or the mass ratio of PVPk12 to nimodipine is 1-200:1, preferably 1-100:1, further preferably 10-50:1, and even further preferably 15-25:1.

5. The nimodipine composition according to claim 3, wherein the mass ratio of sulfobutyl-β-cyclodextrin:PVPk12:nimodipine is 100-300:10-30:1, preferably 150-300:10-50:1, further preferably 150-300:15-25:1, and even further preferably 150-200:15-25:1, or 150-200:15-25:1.

6. A preparation method for nimodipine for injection, wherein the nimodipine for injection is prepared from the composition according to claim 1 and a solvent, and the solvent is selected from ethanol and/or water.

7. The preparation method according to claim 6, comprising the following steps:
1) weighing 1 part of nimodipine, and dissolving in 500-1000 parts of ethanol to obtain an ethanol solution of nimodipine;
2) weighing 100-300 parts of sulfobutyl-β-cyclodextrin and 0-200 parts of stabilizer 2, and dissolving in 1000 parts of water to obtain an aqueous solution of sulfobutyl-β-cyclodextrin;
3) mixing the ethanol solution of nimodipine and the aqueous solution of sulfobutyl-β-cyclodextrin, stirring at an appropriate temperature to obtain clear intermediate 1; and
4) subjecting the clear intermediate 1 to rotary evaporation to remove ethanol, adjusting the concentration to obtain clear intermediate 2, filtering, packing, freeze-drying, and encapsulating to obtain nimodipine for injection.

8. The preparation method according to claim 7, wherein the stirring temperature in step 3) is 30-80° C., preferably 40-80° C., and further preferably 50-60° C.

9. The preparation method according to claim 7, wherein the ethanol concentration in the clear intermediate 2 in step 4) is 0.1-1%.

10. A nimodipine formulation for injection prepared by the preparation method according to claim 7.
